(19) United States
(12) Patent Application Publication
(10) Pub. No.: US 2026/0266843 A1
(43) Pub. Date: Sep. 10, 2026

(54) METHODS FOR DIAGNOSING ADVANCED LIVER FIBROSIS OR LIVER CIRRHOSIS

(71) Applicant: GENFIT, LOOS (FR)

(72) Inventors: ZOUHER MAJD, ENNETIERES-EN-WEPPES (FR); MORGANE DEHORNOIS, LILLE (FR); ALEXANDRA CARON, ALLENNES-LES-MARAIS (FR); YACINE HAJJI, LILLE (FR); JÉRÉMY MAGNANENSI, LILLE (FR); CHRISTIAN ROSENQUIST, HELLERUP (DK)

(21) Appl. No.: 18/993,627

(22) PCT Filed: Jul. 12, 2023
(86) PCT No.: PCT/EP2023/069316

§ 371 Date: Jan. 13, 2025

(30) Foreign Application Priority Data
Jul. 13, 2022 (EP) 22306063.3

(51) Int. Cl. G01N 33/68; A61K 31/216; A61K 31/4178; A61K 31/428; A61K 31/4439; A61K 31/53; A61K 31/575; A61K 31/70; A61K 31/7048; A61K 38/18; A61K 38/26

(52) U.S. Cl. CPC G01N 33/6893; A61K 31/216; A61K 31/4178; A61K 31/428; A61K 31/4439; A61K 31/53; A61K 31/575; A61K 31/70; A61K 31/7048; A61K 38/1825; A61K 38/26; G01N 2333/70542; G01N 2800/085; G01N 2800/52

(57) ABSTRACT

Provided herein is a method of diagnosing or prognosing advanced liver fibrosis or liver cirrhosis in a patient. The method is accomplished by determining a score based on the circulating levels in serum of two markers.

The present invention relates to a method for the diagnosis of advanced liver fibrosis or liver cirrhosis, for prognosing or monitoring the progression of liver fibrosis in a subject or for assessing the efficacy of an anti-fibrotic agent. The invention also relates to a kit for implementing the method of the invention and the compounds for use in the treatment of liver fibrosis, wherein the subject to be treated is identified according to the method of the invention.

### BACKGROUND

Liver fibrosis, common to liver damage and liver diseases, may have many, chronic or not, etiologies including viral Hepatitis B and C (HBV and HCV), Human Immunodeficiency Virus (HIV) and Hepatitis C Virus (HCV) co-infection, Drug-Induced Liver Injury (DILI), cholestatic liver diseases including Primary Biliary Cholangitis (PBC) and Primary Sclerosing Cholangitis (PSC), AIH (AutoImmune Hepatitis), biliary atresia, acute liver disease (ALD), Acute Liver Failure (ALF), cirrhosis, Acute on Chronic Liver Failure (ACLF), Wilson disease, Non-Alcoholic Fatty Liver Disease (NAFLD), Non-Alcoholic SteatoHepatitis (NASH), Alcohol Related Liver Disease (ARLD), alcoholic liver disease and hemochromatosis.

Liver problems can be caused by a variety of factors that damage the liver, such as viruses, immune system abnormality, inherited abnormal genes, cancer, alcohol use and obesity. Over time, conditions that damage the liver can lead to scarring (cirrhosis), which can lead to liver failure, a life-threatening condition that demands urgent medical care. Liver failure occurs when large parts of the liver become damaged beyond repair.

Liver fibrosis is an abnormal wound repair process and is characterized by an excessive accumulation of extracellular matrix protein. It is stimulated by chronic inflammation and occurs as a result of the liver healing process when the liver becomes scarred.

The prediction of liver fibrosis is a key step in the assessment and management of patients with liver damage and/or liver disease. Therefore, since the early and precise evaluation of severity and status of liver fibrosis is essential for diagnosis, monitoring and prognosis, a quantitative measurement is crucial to assess disease progression.

Most forms of liver diseases are without symptom until the disease has progressed to a later stage. The early detection of the disease is therefore challenging. The risk of liver-related mortality increases exponentially with increase in fibrosis stage and mortality and morbidity rates increase exponentially once cirrhosis develops. Cirrhosis is the point where the liver is completely scarred and is beyond the self-healing ability.

Cirrhosis is a leading cause of mortality and morbidity across the world. It is the 11th leading cause of death and 15th leading cause of morbidity, accounting for 2.2% of deaths and 1.5% of disability-adjusted life years worldwide in 2016. Chronic liver diseases caused 1.32 million deaths in 2017 in the world (Sepanlou, et al. The global, regional, and national burden of cirrhosis by cause in 195 countries and territories, 1990-2017: a systematic analysis for the Global Burden of Disease Study 2017. Lancet Gastroenterol Hepatol 2020; 5:245-266.). Cirrhosis is said compensated when patients do not have any visible symptoms of the disease, and cirrhosis is decompensated when cirrhosis has progressed to the point that the liver is having trouble functioning and with the occurrence of symptoms of the disease. Although the clinical features of cirrhosis decompensation are well described (i.e., ascites, spider naevi, jaundice, signs of hepatic encephalopathy), patients who have compensated cirrhosis often have no clinical signs and might be entirely asymptomatic.

Hepatologists and healthcare providers have proposed scoring systems for staging liver fibrosis like the BRUNT/KLEINER system, wherein:
F0 refers to a subject with an absence of liver fibrosis;
F1 refers to a subject with portal or perisinusoidal fibrosis;
F2 refers to a subject with portal/periportal and perisinusoidal fibrosis;
F3 refers to a subject with septal or bridging liver fibrosis;
F4 refers to a subject with liver cirrhosis.

Since severe (fibrosis stage F>2) liver diseases can progress to hepatocellular carcinoma, the accurate staging of liver fibrosis in these liver diseases, especially the early diagnosis of advanced liver fibrosis (Fibrosis stage F3 or F4) and liver cirrhosis (Fibrosis stage F4) is crucial.

Furthermore, the rate of fibrosis progression evolves over time and the diagnostic assay has to be performed several times. In consequence, this test must be repeatable and without risk for the patients, reliable and accurate. Therefore, non-invasive assays are needed for the diagnosis of advanced liver fibrosis (F3 or F4) and liver cirrhosis (F4). It is also important to specifically distinguish patients with cirrhosis (F=4) among patients with severe liver disease, because these F4 patients require an emergency treatment.

So far, invasive liver biopsy remains the Gold Standard for the assessment of liver fibrosis.

Nevertheless, liver biopsy has several recognized limitations including sampling errors, inter-observer variability, and hospitalization. The main disadvantage is the significant risk of complications including bleeding, pain and even death. Furthermore, a biopsy does not reflect the changes in the whole liver and does not differentiate early cirrhosis from progressed cirrhosis and therefore does not constitute a reliable prognostic predictor (Sumida Y et al. Limitations of liver biopsy and non-invasive diagnostic tests for the diagnosis of nonalcoholic fatty liver disease/nonalcoholic steatohepatitis. World J Gastroenterol 2014; 20:475-485).

To avoid above detailed life-threatening risks and diagnostic weakness, in vitro non-invasive diagnostic methods using biomarkers, scores, and physical methods have been developed. By contrast to biopsy which cannot be repeated without inconvenience, these methods can capture the dynamic process of fibrosis resulting from progression and regression since the measures are repeatable. These methods are based on readily available biochemical data and clinical features, such as the FIB4 test (Sterling R K et al. Development of a simple noninvasive index to predict significant fibrosis in patients with HIV/HCV coinfection. Hepatology 2006; 43:1317-1325), or assays that directly measure markers of fibrogenesis and fibrolysis, such as the Enhanced Liver Fibrosis (ELF) test (Guha, I. N., et al., Noninvasive markers of fibrosis in nonalcoholic fatty liver disease: Validating the European Liver Fibrosis Panel and exploring simple markers. Hepatology 2008; 47:455-460).

However, the substitution of these methods for liver biopsy still remains controversial and is not generally accepted due to insufficient diagnostic performance. The main issue of these two biochemical assays is that in individual patients, they cannot reliably differentiate in individual patients the advanced stages of liver fibrosis. Moreover, ELF test is expensive, which constitutes a drawback in case of repetition of the tests. Additionally, FIB4 has a poor performance in patients of less than 35 years old and is less specific in patients over 65.

Physical methods include imaging based on high frequency sound waves (ultrasound and echocardiography), computed tomography (CT), magnetic resonance imaging (MRI), transient elastography (TE, FibroScan) as well as scintigraphy. The main drawbacks of physical measures are the high cost, the low availability of equipment and the complexity of the method that limits the daily clinical practice.

Moreover, obesity, ascites, acute inflammation, liver congestion, and elevated portal vein pressure may for example reduce ultrasound TE (Fibroscan) accuracy by influencing the velocity of shear wave. Furthermore, a falsely increased liver stiffness, due to postprandial increase in portal vein pressure, has been observed with this method.

Therefore, there is still an unmet medical need to develop new non-invasive diagnostic methods having an optimal diagnostic accuracy compared to biopsy, but also useful in monitoring the time course of liver fibrosis and/or showing whether there is a response to a given medication. In addition, it is important to provide a new method giving the best predictivity at the lowest cost and the easiest feasibility.

### SUMMARY OF THE INVENTION

The Inventors have conducted several very fine and complete analysis of a cohort of 1063 patients suffering from NASH and fibrosis to provide novel and highly sensitive non-invasive diagnostic and monitoring methods of advanced liver fibrosis and liver cirrhosis. The inventors have identified a combination of two biological markers, i.e. Soluble Vascular Cell Adhesion Molecule-1 (sVCAM) and Thrombospondin 2 (TSP-2). This new combination of biomarkers provides an accurate diagnosis or prognosis of advanced liver fibrosis (F3 or F4) and liver cirrhosis (F4) compared to existing solutions and represents a valuable alternative to liver biopsy. The measure of these markers in biological fluid samples allows a safe and regular follow up of the liver fibrosis. Furthermore, the diagnostic method of the invention is more accurate and cheaper than methods currently used like ELF and FIB4 in the diagnosis of advanced liver fibrosis and liver cirrhosis.

Accordingly, a first aspect of the invention relates to an in vitro method for diagnosing advanced liver fibrosis (F3 or F4) or liver cirrhosis (F4) in a subject, comprising:
a) measuring the circulating levels of Soluble Vascular Cell Adhesion Molecule (sVCAM) and Thrombospondin 2 (TSP-2) in a biological fluid sample isolated from said subject;
b) comparing the levels of sVCAM and TSP-2 with reference levels of sVCAM and TSP-2, wherein the comparison between measured levels and reference levels is indicative of the presence or absence of advanced liver fibrosis or liver cirrhosis.

In a particular embodiment, the invention relates to an in vitro method for prognosing advanced liver fibrosis (F3 or F4) or liver cirrhosis (F4) in a subject, comprising:
a) measuring the circulating levels of Soluble Vascular Cell Adhesion Molecule (sVCAM) and Thrombospondin 2 (TSP-2) in a biological fluid sample isolated from said subject;
b) comparing the levels of sVCAM and TSP-2 with reference levels of sVCAM and TSP-2, wherein the comparison between measured levels and reference levels is indicative of the presence or absence of advanced liver fibrosis or liver cirrhosis.

In a particular embodiment, step (b) of the method comprises comparing a score A (SA) with a cut-off value, wherein said SA is obtained from the levels of sVCAM and TSP-2 measured in step (a) and the cut-off value is obtained from reference levels of sVCAM and TSP-2. Particularly, said SA and cut-off values are calculated using an algorithm found using a logistic regression function. More particularly, the cut-off value is obtained from Youden' statistical analysis on a training population.

In a more particular embodiment, the SA is calculated through the following algorithm equation:

$$Score = {e}^{y} / ( 1 + {e}^{y} ) with$$

$$y = k + a \times A + b \times B$$

A is the level of sVCAM in log 10 ng/ml;
B is the level of TSP-2 in log 10 ng/ml;
k is the constant of the algorithm equation;
a is a coefficient associated to the level of sVCAM;
b is a coefficient associated to the level of TSP-2;
and wherein:
k is a number comprised between −37.23 and −27.54, in particular-30.000;
a is a number comprised between 5.432 and 8.646, in particular 7.250;
b is a number comprised between 5.746 and 7.727, in particular 6.921; and

In this particular aspect of the invention, the Score corresponds to SA in the above algorithm equation.

In a particular embodiment, a SA higher than a cut-off value co1 is indicative of advanced liver fibrosis, particularly co1 being comprised between 0.200 and 0,500, more particularly co1 may be 0.204, 0.3383, 0.4852.

More particularly co1 is equal to 0.3383.

In another particular embodiment, the SA higher than a cut-off value co2 is indicative of liver cirrhosis, particularly co2 being comprised between 0.510 and 0.790, more particularly co2 may be 0.5110, 0.6073, 0.7791, More particularly, co2 is equal to 0.6073.

Another aspect of the invention relates to an in vitro method for monitoring the progression of liver fibrosis in a subject, comprising the steps of:
a) measuring the circulating levels of sVCAM and TSP-2 in a biological fluid sample isolated from said subject, and
b) comparing said levels with levels of sVCAM and TSP-2 previously measured in the same subject.

In a particular embodiment, step (b) of said method comprises comparing a score C (SC) to a score B (SB), wherein SC is a score obtained from the levels of sVCAM and TSP-2 measured in step (a) and SB is a score obtained from levels of sVCAM and TSP-2 previously measured, said scores being calculated by using an algorithm equation found using a logistic regression. In a particular embodiment, an increase of SC compared to SB indicates the progression of liver fibrosis. In another particular embodiment, a decrease of SC compared to SB indicates the regression of liver fibrosis. In a specific embodiment, no significant change between SC and SB measured in a certain lapse of time in a same subject means that liver fibrosis is stable.

In a particular embodiment of said method, step (a) of said method for monitoring the progression of liver fibrosis is implemented at least 3 months after a previous measurement of the levels of sVCAM and TSP-2, particularly in a period between 3 months and 10 years, more particularly in a period between 3 months and 2 years, after a previous measurement of the levels of sVCAM and TSP-2.

In a specific embodiment of said method, one event linked to the evolution of pathological state occurs during the monitoring, between SB and SC. In a particular embodiment, said event is selected from liver transplantation, acute on chronic liver fibrosis, compensated cirrhosis, decompensated cirrhosis, episode of ascites and presence of esophageal varices at endoscopy.

In a preferred embodiment, said SB and SC are calculated through the above defined algorithm equation.

A third aspect of the invention concerns a method for assessing the efficacy of an anti-fibrotic agent in treating advanced liver fibrosis or liver cirrhosis, comprising
a) measuring the circulating levels of sVCAM and TSP-2 in a biological fluid sample isolated from a subject suffering from advanced liver fibrosis, wherein said subject has been administered an anti-fibrotic agent before said measure, and
b) comparing said levels of sVCAM and TSP-2 with the levels of sVCAM and TSP-2 previously measured before administration of the anti-fibrotic agent to the same subject to assess the efficacy of said anti-fibrotic agent.

In a particular embodiment of this method, step (b) comprises comparing a score E (SE) to a score D (SD), wherein SD is a score obtained from the levels of sVCAM and TSP-2 measured before administration of an anti-fibrotic agent to the subject and SE is a score obtained from the levels of sVCAM and TSP-2 measured in step (a), i.e. after administration of an anti-fibrotic to the subject, SE and SD being calculated through an algorithm. Particularly, a decrease of SE compared to SD indicates the efficacy of the anti-fibrotic agent, an increase of SE compared to SD indicates the non-efficacy of the anti-fibrotic agent, and/or the non-responsiveness of the patient.

In a preferred embodiment, said SE and SD are calculated through the above defined algorithm equation.

In a preferred embodiment, the biological fluid sample of the subject used in the methods of the present invention is an interstitial fluid, saliva, urine or whole blood sample. In a particular embodiment, the biological fluid sample of the subject used in the methods of the present invention is a blood sample. Preferably, the biological fluid sample is cell-free. More preferably, the blood sample is a plasma or serum sample from a subject.

Another aspect of the invention is also to provide anti-fibrotic agents for use in the treatment of advanced liver fibrosis or liver cirrhosis in a subject, wherein said subject is diagnosed as suffering from advanced liver fibrosis or liver cirrhosis according to the method of the present invention, wherein said agent is selected in the group consisting of pegbelfermin, cenicriviroc, dapagliflozin, dulaglutide, empagliflozin, fenofibrate, lanifibranor, liraglutide, obeticholic acid, pioglitazone, resmetirom, saroglitazar magnesium, seladelpar, semaglutide, sitagliptin, TERN-101, TERN-201, tropifexor, ambrisentan, BMS-963272, BMS-986251, BMS-986263, HepaStem, LYS006, MET409, MET642 and orlistat.

The present invention also provides a kit for diagnosing advanced liver fibrosis or liver cirrhosis in a subject, said kit comprising means for determining the levels of sVCAM and TSP-2. In a particular embodiment, said means are an antibody or aptamer or peptide directed against sVCAM and an antibody or aptamer or peptide directed against TSP-2.

### DETAILED DESCRIPTION OF THE INVENTION

### Definitions

### Fibrosis Scoring/Staging

According to the present invention, the term “fibrosis” or “liver fibrosis” denotes a pathological condition of excessive deposition of fibrous connective tissue in the liver. More specifically, fibrosis is a pathological process, which includes a persistent fibrotic scar formation and overproduction of extracellular matrix by the connective tissue, as a response to tissue damage. Physiologically, the deposit of connective tissue can obliterate the architecture and function of liver.

The different stages of liver fibrosis are defined by the Kleiner scoring system (Kleiner et al, Hepatology 2005, Vol 41, Issue 6, 1313-1321) wherein:
F0 refers to a subject with an absence of liver fibrosis;
F1 refers to a subject with portal or perisinusoidal fibrosis;
F2 refers to a subject with portal/periportal and perisinusioidal fibrosis;
F3 refers to a subject with septal or bridging liver fibrosis;
F4 refers to a subject with liver cirrhosis.

The stage of F0-2 is assigned to subjects having early liver fibrosis, F3 or F4 is assigned to subjects having advanced liver fibrosis, and the stage F4 is assigned to subjects having liver cirrhosis.

Using this fibrosis staging system, patients with no or minimal fibrosis (F=0 or 1) are generally not considered at risk of cirrhosis, liver failure, HCC (hepatocellular carcinoma) or liver-related death. Patients with severe (F>2) liver fibrosis are at risk of developing cirrhosis, liver failure, HCC and liver-related death. Patients with compensated cirrhosis (F=4) are at high risk of liver failure (decompensated cirrhosis), HCC and liver-related deaths.

In the context of the present invention, the term “advanced fibrosis” or “advanced liver fibrosis” refers to a fibrosis stage of F≥3, i.e. a fibrosis stage F3 or a fibrosis stage F4.

In the context of the present invention, the term “cirrhosis” or “liver cirrhosis” refers to a fibrosis stage of F4.

The terms “subject” and “patient” may be used interchangeably herein and refer to a human subject.

Within the context of the present invention, the terms “biomarker”, “marker”, “biological marker” are interchangeable.

Within the scope of the present invention, any range must be considered as including the upper and lower limits.

### Concurrent Noninvasive Tests

The Fibrosis-4 (FIB-4) index is calculated as age (years)×AST(U/L)/platelet(×10⁹/L)/√ALT(U/L), where AST is aspartate aminotransferase and ALT is alanine aminotransferase.

The Enhanced Liver Fibrosis panel (ELF, Siemens Healthcare GmbH, Eriangen, Germany) is a test to predict fibrosis based on three fibrosis biomarkers: hyaluronic acid (HA), tissue inhibitor of matrix metalloproteinases-1 (TIMP-1) and amino-terminal propeptide of procollagen type III (PIIINP). This test uses the following equation to calculate the ELF score (2.278+0.851 ln(_{CHA})+0.751 ln(_{CPINP})+0.394 ln(_{CTIMP-1})), wherein C is the concentration of the biomarker.

### Diagnostic Performance Analysis

Sensitivity is the ability of a test to correctly identify those who have the disease. The sensitivity measures the proportion of positive (“disease”) cases in a population of sick patients evaluated using a reference method (“gold standard”), or as reliable as possible considering biopsy profiling. Sensitivity (Se) is the proportion of positive results (True Positive=TP) divided by the total number of sick patients (TP+False Negative=FN): Se=TP/(TP+FN). Sensitivity is usually expressed as a percentage (%), from 0 to 100%.

True Positive (TP) subjects are subjects with the disease with the value of a parameter of interest above a cut-off.

False Negative (FN) subjects are subjects with the disease with the value of a parameter of interest below a cut-off.

Specificity is the ability of a test to correctly identify those who do not have the disease. The specificity measures the proportion of negative (“healthy”) cases in a population of healthy patients evaluated using a reference method (“gold standard”). Specificity (Sp) is the proportion of negative results (True Negative=TN) divided by the total number of healthy patients (TN+False Positive=FP):Sp=TN/(TN+FP).

True Negative (TN) subjects are subjects without the disease with the value of a parameter of interest below a cut-off.

False Positive (FP) subjects are subjects without the disease with the value of a parameter of interest above a cut-off.

The prevalence of a given population is the number of cases of the disease within the population.

The Positive Predictive Value (PPV) is the probability to have the disease when a test is positive. PPV=number of True Positives/(number of True Positives+number of False Positives). PPV=TP/(TP+FP). The negative Predictive Value (NPV) is the probability of not having the disease when a test is negative. NPV=number of True Negatives/(number of True Negatives+number of False Negatives). NPV=TN/(TN+FN).

Prevalence affects PPV and NPV differently. PPV is increasing, while NPV decreases with the increase of the prevalence of the disease in a population. Whereas the change in PPV is more substantial, NPV is somewhat less influenced by disease prevalence. For low prevalence (5-10%), PPV value is low. In parallel, NPV is high. For high prevalence (80-90%), PPV value is high and vice versa for the NPV.

Likelihood ratio is defined as the ratio of expected test result in subjects with a certain state/disease to the subjects without the disease.

Likelihood ratio for positive test results (LR+) represents the ratio of the probability that the positive test result is to occur in subjects with the disease compared to those without the disease. LR+=sensitivity/(1−specificity). LR+ is the best indicator for ruling-in diagnosis. The higher the LR+, the more the test is indicative of a disease. LR+>5 indicates a moderate to large increase evidence that the disease is present. Good diagnostic tests have LR+>10 and their positive result has a significant contribution to the diagnosis.

Likelihood ratio for negative test result (LR−) represents the ratio of the probability that a negative result will occur in subjects with the disease to the probability that the same result will occur in subjects without the disease. LR−=(1−sensitivity)/specificity. LR− is a good indicator for ruling-out the diagnosis. LR-between 0.1 and 0.2 indicates a moderate probability to well diagnose patients without the studied condition. Good diagnostic test of patients that are without the studied condition has LR−<0,1.

There is a pair of diagnostic sensitivity and specificity values for every individual cut-off. To construct a receiver operating characteristic (ROC) graph, these pairs of values are plotted on the graph with the 1-specificity on the x-axis and sensitivity on the y-axis. The shape of a ROC curve and the area under the receiver operating characteristic curve (AUROC) shows how high is the discriminative power of a test. The closer the curve is located to upper-left hand corner and the larger the area under the curve, the better the test is at discriminating between diseased and non-diseased subjects. The area under the curve can have any value between 0.5 and 1 and it is a good indicator of the goodness of the test. A perfect diagnostic test has an AUROC of 1.0. whereas a non-discriminating test has an AUROC of 0.5. AUROC is a global measure of diagnostic accuracy.

### Method of the Invention

As mentioned above, advanced liver fibrosis and cirrhosis are associated with liver-related death and easy detection of advanced fibrotic subjects and cirrhotic subjects is thus of outmost importance. The present invention provides a solution to these unmet needs.

In the methods of the present invention, the levels of two circulating markers are measured from a blood, serum or plasma sample from a subject. Said 2 circulating markers are: Soluble Vascular Cell Adhesion Molecule-1 (sVCAM) and Thrombospondin 2 (TSP-2). sVCAM is also known as VCAM 1, INCAM-100, CD106 and registered in database UniProt under the number P19320. TSP-2 is also known as THBS2 and registered in database UniProt under the number P35442.

In a particular embodiment, the invention relates to an in vitro method for diagnosing or prognosing advanced liver fibrosis or liver cirrhosis in a subject, comprising
a) measuring the circulating levels of sVCAM and TSP-2 in a biological fluid sample isolated from said subject;
b) comparing the levels of sVCAM and TSP-2 with reference levels of sVCAM and TSP-2, wherein the comparison between measured levels and reference levels is indicative of the presence or absence of advanced liver fibrosis or liver cirrhosis.

The measure of the circulating levels of sVCAM and TSP-2, is conducted in a biological fluid sample of a subject. In all the methods and embodiments presented herein, the biological fluid sample may be a sample of blood or of a blood-derived fluid such as serum and plasma, of saliva, of interstitial fluid or of urine. In a particular embodiment, the biological fluid sample of the subject is a blood, serum, or plasma sample. In a more particular embodiment, the sample is a serum sample. In another particular embodiment, the biological fluid sample is a cell-free sample.

The circulating levels of sVCAM and TSP-2 may be measured by any conventional methodology well known in the art, such as immunoassays (e.g. ELISA (enzyme-linked immunosorbent assay), immunoturbidimetry, immuno-nephelometry, immune cytometry, protein array). For example, the levels of sVCAM and TSP-2 can be determined by antibodies, aptamers or peptides respectively directed against said markers.

The “reference levels” of sVCAM and TSP-2 may be measured from a group of healthy subjects and/or from a group of subjects who are diagnosed as suffering early liver fibrosis and/or a group of subjects who are diagnosed as suffering from advanced liver fibrosis but without liver cirrhosis and/or a group of subjects who are diagnosed as suffering from liver cirrhosis. In a particular embodiment, the reference levels of sVCAM and TSP-2 are measured from a “training population”.

“Training population” refers to a population consisted of a given number of subjects, wherein the fibrosis stage of each subject is already determined by a method of prior art, like liver biopsy.

In a particular embodiment, the circulating levels of sVCAM and TSP-2 are compared with specific positive controls, which are used to validate the measures. Said positive controls respectively contain different concentrations of sVCAM and TSP-2 that correspond to the ranges of these markers measured in a training population. For instance, the positive controls for TSP-2 may comprise from 11 to 300 ng/ml of TSP-2, and the positive controls for sVCAM may comprise from 390 to 6190 ng/ml of sVCAM.

In a particular embodiment, the values of levels of circulating markers sVCAM and TSP-2 measured in the biological fluid sample of the subject can be introduced into a mathematical function (i.e. a statistical algorithm) to obtain a score A (SA). Said score can accurately predict advanced liver fibrosis or liver cirrhosis, respectively according to a specific cut-off value.

By being compared to a specific cut-off value, the SA thus can be used to discriminate subjects having advanced liver fibrosis or liver cirrhosis from subjects not having advanced liver fibrosis, or not having liver cirrhosis. One skilled in the art is aware of numerous suitable methods for developing mathematical function, and all of these are within the scope of the present invention. In a particular embodiment, the mathematical function includes a logistic regression equation.

In a particular embodiment, in step (b) of the above method, a score SA is compared to a cut-off value, said SA being obtained from the levels of sVCAM and TSP-2 measured in step (a), said cut-off value being obtained from reference levels of sVCAM and TSP-2, said SA and cut-off value being calculated using an algorithm equation.

According to an embodiment of the invention, SA value is compared to a cut-off value (co). Said value may be calculated from Youden' statistical analysis on a training population. Particularly, said SA and cut-off value are calculated using an algorithm equation to differentiate patients having advanced liver fibrosis from patients without advanced liver fibrosis.

Alternatively, said SA and cut-off values are calculated using an algorithm equation to differentiate patients having liver cirrhosis from patients without liver cirrhosis.

A cut-off value co1 may be determined to be used to indicate the presence or absence of advanced liver fibrosis. A cut-off value co2 may be determined to be used to indicate the presence or absence of liver cirrhosis.

In a more particular embodiment, the invention relates to an in vitro method for diagnosing advanced liver fibrosis in a human subject, comprising
a) measuring the circulating levels of sVCAM and TSP-2 in a biological fluid sample isolated from said human subject,
b) calculating a score SA based on the measures of step (a) by using an algorithm equation,
c) comparing this SA to a cut-off value co1,
d) wherein a SA greater than the cut-off value co1 is indicative of a subject with advanced liver fibrosis.

In contrast, a calculated SA value lower than the cut-off value co1 is indicative of a subject not having advanced liver fibrosis.

In another more particular embodiment, the invention relates to an in vitro method for diagnosing liver cirrhosis in a human subject, comprising
a) measuring the circulating levels of sVCAM and TSP-2 in a biological fluid sample isolated from said human subject,
b) calculating a score SA based on the measures of step (a) by using an algorithm equation,
c) comparing this SA to a cut-off value co2,
d) wherein a SA greater than the cut-off value co2 is indicative of a subject with liver cirrhosis.

In contrast, a calculated SA value lower than the cut-off value co2 is indicative of a subject not having liver cirrhosis.

To find the algorithm equation that respectively separates the patients with advanced liver fibrosis from patients without advanced liver fibrosis and the patients with liver cirrhosis from patients without liver cirrhosis, a correlation analysis was done to cluster covariates that were highly correlated (Pearson coefficient≥0·5). The most discriminating variables of each group, determined by computing p values from Student's t test, were retained. Thus, a set of variables were selected to avoid high multicollinearity issues. This subset of variables was analyzed by running logistic regression analyses, and those significantly associated with advanced fibrosis and/or cirrhosis (sVCAM and TSP-2) were retained and their levels values were combined in an algorithm and led to an equation with coefficients (a, b, c) and a constant k.

The algorithm produced a score that ranged from 0 to 1 (continuous).

For each equation, using Excel Solver, has been calculated the Log-Likelihood Function (LL) which is the conditional probability that predicted dependent variable (here are the coefficients a, b, c and the constant k) corresponds to the actual observed value (the disease in the present case; liver fibrosis status) given the values of the independent variables inputs (the value of the markers measured, in the present case).

The conditional probability Pr(Yi=yi|X1i,X2i, . . . . Xki)
where Pr=probability,
yi=coefficients,
X1i, X2i, . . . . Xki are the measured variables,
Y is the dependent variable or the score S. If this score=0 it means that the patient is diagnosed without advanced liver fibrosis or liver cirrhosis and if this score=1 it means that the patient is diagnosed with advanced liver fibrosis or liver cirrhosis, i is any patient.

The conditional probability is abbreviated Pr (Y=y|X) for convenience and is calculated by the following formula:

$$Pr ( Y = y \vert X ) = P ( X ) Y * [ 1 - P ( X ) ] ( 1 - Y )$$

Taking the natural log of both sides yields the following:

$$ln [ Pr ( Y = y \vert X ) ] = y * ln [ P ( X ) ] * ( 1 - y ) * ln [ [ 1 - P ( X ) ] ]$$

The Log-Likelihood Function, LL, is then the sum of the ln[Pr (Y=y|X)] terms for all data records as per the following formula:

$$L L = \sum Yi * P ( Xi ) + ( 1 - Yi ) * ( 1 - P ( Xi ) )$$

The objective of logistic regression is to determine, using Excel Solver, the parameters of the algorithm equation (such as a, b, c, k) that maximize LL (the lowest value of LL) meaning the highest probability that the score calculated by the algorithm corresponds to the actual observed status of the disease.

The subset of 2 variables, i.e. sVCAM and TSP-2, was analyzed by logistic regression (using Excel Solver) to create the following algorithm:

$$Score = {e}^{y} / ( 1 + {e}^{y} )$$

$$Where y = k + a \times ( {Log}_{10} sVCAM [ ng / L ] ) + b \times ( {Log}_{10} TSP - 2 [ ng / mL ] ) .$$

Particularly, said Score is calculated through the following algorithm equation:

$$Score = {e}^{y} / ( 1 + {e}^{y} ) with$$

$$y = k + a \times A + b \times B$$

A is the level of sVCAM in log 10 ng/ml;
B is the level of TSP-2 in log 10 ng/ml;
k is the constant of the algorithm equation;
a is a coefficient associated to the level of sVCAM;
b is a coefficient associated to the level of TSP-2;
and wherein:
k is a number comprised between −37.23 and −27.54, in particular-30.000;
a is a number comprised between 5.432 and 8.646, in particular 7.250;
b is a number comprised between 5.746 and 7.727, in particular 6.921

In a particular embodiment, the score A (SA), the cut-off value co1 and the cut-off value co2 used for the diagnosis of advanced liver fibrosis or liver cirrhosis are calculated according to above defined algorithm equation on a training population.

By way of example, the following equations may be used for the diagnosis of advanced liver fibrosis or liver cirrhosis:

$$y = - 30.000 + 7.250 \times log 10 ( sVCAM ( ng / L ) ) + 6.921 \times log 10 ( TSP - 2 ( ng / mL ) )$$

$$y = - 34 + 8 \text{.6} \times log 10 ( sVCAM ( ng / L ) ) + 6.7 \times log 10 ( TSP - 2 ( ng / mL ) )$$

$$or$$

$$y = - 30.001 + 8.64 \times log 10 ( sVCAM ( ng / L ) ) + 5.75 \times log 10 ( TSP - 2 ( ng / mL ) )$$

$$or$$

$$y = - 27.2 + 5.9 \times log 10 ( sVCAM ( ng / L ) ) + 7.7 \times log 10 ( TSP - 2 ( ng / mL ) )$$

$$or$$

$$y = - 31.2 + 5.91 \times log 10 ( sVCAM ( ng / L ) ) + 7.71 \times log 10 ( TSP - 2 ( ng / mL ) )$$

$$or$$

$$y = - 30.012 + 6.6 \times log 10 ( sVCAM ( ng / L ) ) + 7.69 \times ( TSP - 2 ( ng / mL ) )$$

$$y = - 30.000 + 7.250 \times log 10 ( sVCAM ( ng / L ) ) + 6.921 \times log 10 ( TSP - 2 ( ng / mL ) )$$

$$or$$

$$y = - 30.001 + 8.64 \times log 10 ( sVCAM ( g / L ) ) + 5.75 \times log 10 ( TSP - 2 ( ng / mL ) )$$

are preferred.

In a particular embodiment, SA is compared with the cut-off value co1, which is indicative of an advanced liver fibrosis. Particularly, co1 is comprised between 0.200 and 0.500, more particularly between 0.2040 and 0.4852, in particular equal to 0.3383.

In another specific embodiment, SA is compared with the cut-off value co2, which is indicative of a liver cirrhosis. Particularly, co2 is comprised between 0.510 and 0.790, more particularly between 0.5110 and 0.7791 in particular equal to 0.6073.

According to another aspect, the invention relates to a computer program comprising instructions that, when executed by a processor/processing means, cause the processor/processing means to:
receive measured levels of sVCAM and TSP-2;
calculate SA from these measured levels, from a mathematical function as described herein; and
assign the subject into the group of subjects having advanced liver fibrosis or liver cirrhosis upon the calculated score compared to predetermined cut-off values.

The present invention further provides a computer readable medium comprising the computer program described therein. According to a particular embodiment, the computer readable medium is non-transitory medium or a storage medium.

The present invention also provides an in vitro method for monitoring the progression of liver fibrosis in a subject by measuring the levels of two circulating markers, i.e. sVCAM and TSP-2.

Particularly, said method comprises the steps of:
a) measuring the circulating levels sVCAM and TSP-2 in a biological fluid sample isolated from said subject, and
b) comparing said levels with levels of sVCAM and TSP-2 previously measured in the same subject.

Circulating levels of sVCAM and TSP-2 measured in step (a) may be introduced into an algorithm equation as defined above, to calculate a score. Since said score is calculated by using a non-invasive method and correlated with the evolution of liver fibrosis, it allows not only to diagnose but also to monitor liver fibrosis progression, by repeated measures.

In a particular embodiment, the circulating levels of sVCAM and TSP-2 are measured from one or more blood-derived sample(s) from the subject. In that case, the same kind of sample is used each time a measure has to be done. For the sake of clarity, this means that if a previous measure was done from a serum sample, the subsequent measures are done from serum samples of the same subject. Likewise, if the previous measure was done from a blood or plasma sample, the subsequent measures are done from blood or plasma samples, respectively, of the same subject. In a particular embodiment, the circulating levels of the markers are measured from one or more serum sample(s) from the subject.

The collection over time of several samples from the same subject allows to assess longitudinal changes in the score. For instance, a first score, named SB, may be calculated from the levels of sVCAM and TSP-2 previously measured in a biological fluid sample of a subject, and a second score, named SC, may be calculated from the levels sVCAM and TSP-2 subsequently measured in a biological fluid sample of the same subject. SB and SC may be obtained through the algorithm equation as defined above. If the score increases with time in the same subject, i.e. SC is higher than SB, it means that liver fibrosis worsens, whereas if the SC decreases with time in the same subject, i.e. SC is lower than SB, it means that liver fibrosis decreases. No significant change between SC and SB measured in a certain lapse of time in a same subject means that liver fibrosis is stable.

During a follow-up, the change in the value between SC and SB is therefore an indicator of liver fibrosis progression or liver fibrosis regression.

A particular embodiment, said method comprises the steps of:
a) measuring the circulating levels sVCAM and TSP-2 in a biological fluid sample isolated from said subject to obtain a score C, and
b) comparing said score C with a score B obtained with levels of sVCAM and TSP-2 previously measured in the same subject.

Since the score calculated according to the algorithm equation as defined above is a linear value, by comparing the score C (SC) with the score B (SB), the method of the invention also allows to determine the likelihood of a subject with advanced liver fibrosis to progress towards cirrhosis or towards worsening cirrhosis.

In a particular embodiment, SC is measured while an event linked to the evolution of pathological state occurs. Said events comprise liver transplantation, acute on chronic liver fibrosis (ACLF), compensated cirrhosis and decompensated cirrhosis, episode of ascites and presence of esophageal varices at endoscopy. Preferably, the events are acute on chronic liver fibrosis, compensated and decompensated cirrhosis.

In a particular embodiment, the SC is measured at least 3 months after the measurement of SB, particularly in a period between 3 months and 10 years, preferably in a period between 3 months and 2 years, more preferably between 1 and 2 years, after the measurement of SB.

In a more particular embodiment, if the subject is diagnosed as having liver cirrhosis, e.g. by the method of the invention as described above, then SC is measured in a period between 3 months and 2 years, and preferentially 3 months, after the measurement of SB. The suitable moment for the measurement of SC may depend on comorbidities.

Comorbidities comprise malignancy, type 2 diabetes, overweight and obesity, heart disease and kidney diseases.

In another particular embodiment, if the subject is diagnosed as having advanced liver fibrosis, e.g. by the method of the invention as described above, then SC is measured in a period between 1 and 10 years after the measurement of SB. The suitable moment for the measurement of SC depends on comorbidities.

Thanks to the methods of the invention, a decision may be taken to give life-style recommendations to a subject (such as a food regimen or providing physical activity recommendations), to medically take care of a subject (e.g. by setting regular visits to a physician or regular examinations, for example for regularly monitoring markers of liver damage), or to administer at least one liver fibrosis therapy to the patient, to treat advanced liver fibrosis or liver cirrhosis. Particularly, a decision may be taken to give life-style recommendations to a subject or to administer at least one liver fibrosis therapy. The invention thus further relates to an anti-fibrotic compound for use in a method for treating advanced liver fibrosis or liver cirrhosis in a subject in need thereof, wherein the subject has been identified thanks to a method according to the invention.

The invention thus further relates to an anti-fibrotic compound for use in a method for treating advanced liver fibrosis or liver cirrhosis in a subject in need thereof, wherein the subject has been identified thanks to a method according to the invention.

The term “treatment”, as used herein, relates to both therapeutic measures and prophylactic or preventive measures, wherein the goal is to prevent or slow down (lessen) an undesired physiological change or disorder. Beneficial or desired clinical results include, but are not limited to, alleviation of symptoms, stabilizing pathological state (specifically not worsening), slowing down or stopping the progression of the disease, improving or mitigating the pathological condition. Particularly, for the purpose of the present invention, treatment is directed to slow the progression of fibrosis and reduce the risk of further complications. It can also involve prolonging survival in comparison with the expected survival if the treatment is not received.

The anti-fibrotic agent is administered in a therapeutically effective amount. As used herein, the expression “therapeutically effective amount” refers to an amount of the drug effective to achieve a desired therapeutic result. A therapeutically effective amount of a drug may vary according to factors such as the disease state, age, sex, and weight of the individual, and the ability of drug to elicit a desired response in the individual. A therapeutically effective amount is also one in which any toxic or detrimental effects of agent are outweighed by the therapeutically beneficial effects. The effective dosages and dosage regimens for drug depend on the disease or condition to be treated and may be determined by the persons skilled in the art. A physician having ordinary skill in the art may readily determine and prescribe the effective amount of the pharmaceutical composition required. For example, the physician could start doses of drug employed in the pharmaceutical composition at levels lower than that required to achieve the desired therapeutic effect and gradually increase the dosage until the desired effect is achieved. In general, a suitable dose of a composition of the present invention will be that amount of the compound which is the lowest dose effective to produce a therapeutic effect according to a particular dosage regimen. Such an effective dose will generally depend upon the factors described above.

The invention further relates to an anti-fibrotic compound for use in a method for treating liver fibrosis in a F3 or F4 patient, wherein the patient is classified as having advanced liver fibrosis or liver cirrhosis according to the method of the invention. The invention also relates to an anti-fibrotic compound for use in a method for treating liver fibrosis, wherein the subject, diagnosed or classified as having advanced liver fibrosis or liver cirrhosis, is treated with an anti-fibrotic compound as defined herebelow, thanks to a method according to the invention.

Anti-fibrotic compounds comprise:
a compound of formula (I) or a pharmaceutically acceptable salt thereof:

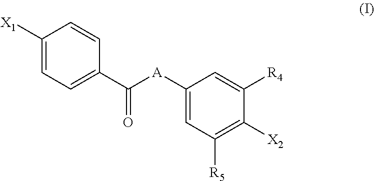

wherein:
X1 represents a halogen atom, a R1 group or G1-R1 group;
A represents a CH═CH or CH2-CH2 group;
X2 represents a G2-R2 group;
G1 represents an atom of oxygen;
G2 represents an atom of oxygen or sulfur;
R1 represents a hydrogen atom, an unsubstituted alkyl group, an aryl group or an alkyl group that is substituted by one or more substituents selected from halogen atoms, alkoxy groups, alkylthio groups, cycloalkyl groups, cycloalkylthio groups and heterocyclic groups;
R2 represents an alkyl group substituted by a —COOR3 group, wherein R3 represents a hydrogen atom or an alkyl group that is substituted or not by one or more substituents selected from halogen atoms, cycloalkyl groups and heterocyclic groups.
R4 and R5, identical or different, represent an alkyl group that is substituted or not by one or more substituent selected from halogen atoms, cycloalkyl groups and heterocyclic groups;
   AMP activated protein kinase stimulators such as PXL-770, MB-11055, Debio-0930B, metformin, CNX-012, O-304, mangiferin calcium salt, eltrombopag, carotuximab, and imeglimin;
   Bile acids such as obeticholic acid (OCA), ursodeoxycholic acid (UDCA), norursodeoxycholic acid, and ursodiol;
   CCR antagonists such as cenicriviroc (CCR2/5 antagonist), PG-092, RAP-310, INCB-10820, RAP-103, PF-04634817, and CCX-872;
   Dipeptidyl peptidase IV (DPP4) inhibitors such as evogliptin, vidagliptin, fotagliptin, alogliptin, saxagliptin, tilogliptin, anagliptin, sitagliptin, retagliptin, melogliptin, gosogliptin, trelagliptin, teneligliptin, dutogliptin, linagliptin, gemigliptin, yogliptin, betagliptin, imigliptin, omarigliptin, vidagliptin, and denagliptin;
   Farnesoid X receptor (FXR) agonists such as obeticholic acid (OCA), tropifexor (LJN452), cilofexor (GS9674), Nidufexor (LMB763), EDP-305, AKN-083, INT-767, GNF-5120, LY2562175, INV-33, NTX-023-1, EP-024297, Px-103, SR-45023, TERN-101 (6-{4-[5-Cyclopropyl-3-(2,6-dichloro-phenyl)-isoxazol-4-ylmethoxy]-piperidin-1-yl}-1-methyl-1H-indole-3 carboxylic acid), TERN-201, TERN-501 and TERN-301;
   Fibroblast Growth Factor 19 (FGF-19) receptor ligand or functional engineered variant of FGF-19;
   Fibroblast Growth Factor 21 (FGF-21) agonists such as PEG-FGF21 (pegbelfermin, formerly BMS-986036), YH-25348, BMS-986171, YH-25723, LY-3025876, and NNC-0194-0499;
   engineered Fibroblast Growth Factor 19 (FGF-19) analogues such as NGM-282 (aldafermin);
   Glucagon-like peptide-1 (GLP-1) analogs such as semaglutide, liraglutide, exenatide, albiglutide, dulaglutide, lixisenatide, loxenatide, efpeglenatide, taspoglutide, MKC-253, DLP-205, and ORMD-0901;
   Nicotinic acid such as Niacin and Vitamin B3;
   nitazoxanide (NTZ), its active metabolite tizoxanide (TZ) or other prodrugs of TZ such as RM-5061;
   PPAR alpha agonists such as fenofibrate, ciprofibrate, pemafibrate, gemfibrozil, clofibrate, binifibrate, clinofibrate, clofibric acid, nicofibrate, pirifibrate, plafibride, ronifibrate, theofibrate, tocofibrate, and SR10171;
   PPAR gamma agonists such as pioglitazone, deuterated pioglitazone, rosiglitazone, efatutazone, ATx08-001, OMS-405, CHS-131, THR-0921, SER-150-DN, KDT-501, GED-0507-34-Levo, CLC-3001, and ALL-4;
   PPAR delta agonists such as GW501516 (Endurabol or ({4-[{{4-methyl-2-[4-(trifluoromethyl)phenyl]-1,3-thiazol-5-yl}methyl)sulfanyl]-2-methylphenoxy}acetic acid)), MBX8025 (Seladelpar or {2-methyl-4-[5-methyl-2-(4-trifluoromethyl-phenyl)-2H-[1,2,3]triazol-4-ylmethylsylfanyl]-phenoxy}-acetic acid), GW0742 ([4-[[[2-[3-fluoro-4-(trifluoromethyl)phenyl]-4-methyl-5-thiazolyl]methyl]thio]-2-methyl phenoxy]acetic acid), L165041, HPP-593, and NCP-1046;
   PPAR alpha/gamma dual agonists (also named glitazars) such as saroglitazar, aleglitazar, muraglitazar, tesaglitazar, and DSP-8658;
   PPAR gamma/delta dual agonists such as conjugated linoleic acid (CLA), and T3D-959;
   PPAR alpha/gamma/delta pan agonists or PPARpan agonists such as IVA337, TTA (tetradecylthioacetic acid), bavachinin, GW4148, GW9135, bezafibrate, lanifibranor, lobeglitazone, and CS038;
   Sodium-glucose transport (SGLT) 2 inhibitors such as licoglifozin, remogliflozin, dapagliflozin, empagliflozin, ertugliflozin, sotagliflozin, ipragliflozin, tianagliflozin, canagliflozin, tofogliflozin, janagliflozin, bexagliflozin, luseogliflozin, sergliflozin, HEC-44616, AST-1935, and PLD-101.
   stearoyl CoA desaturase-1 inhibitors/fatty acid bile acid conjugates such as aramchol, GRC-9332, steamchol, TSN-2998, GSK-1940029, and XEN-801;
   thyroid receptor β (THR β) agonists such as VK-2809, resmetirom (MGL-3196), MGL-3745, SKL-14763, sobetirome, BCT-304, ZYT-1, MB-07811 and eprotirome;
   Vitamin E and isoforms; vitamin E combined with vitamin C and atorvastatin.

In the present invention, anti-fibrotic compounds are preferably selected in the group consisting of pegbelfermin, cenicriviroc, dapagliflozin, dulaglutide, empagliflozin, fenofibrate, lanifibranor, liraglutide, obeticholic acid, pioglitazone, resmetirom, saroglitazar magnesium, seladelpar, semaglutide, sitagliptin, TERN-101, TERN-201, tropifexor, ambrisentan, BMS-963272, BMS-986251, BMS-986263, HepaStem, LYS006, MET409, MET642, and orlistat (Xenical).

More preferably, the anti-fibrotic agent is selected from pegbelfermin, cenicriviroc, dapagliflozin, dulaglutide, empagliflozin, fenofibrate, lanifibranor, liraglutide, obeticholic acid, pioglitazone, resmetirom, saroglitazar magnesium, seladelpar, semaglutide, sitagliptin, TERN-101, TERN-201 and tropifexor.

The invention further relates to a method for assessing the efficacy of an anti-fibrotic agent in a subject suffering from advanced liver fibrosis or liver cirrhosis, comprising:
a) measuring the circulating levels of sVCAM and TSP-2 in a biological fluid sample isolated from a subject suffering from advanced liver fibrosis, wherein said subject has been administered an anti-fibrotic agent before said measure, and
b) comparing said levels of sVCAM and TSP-2 with the levels of sVCAM and TSP-2 previously measured before administration of the anti-fibrotic agent to the same subject to assess the efficacy of said anti-fibrotic agent.

In a particular embodiment, the measures of levels of sVCAM and TSP-2 respectively before and after an administration of an anti-fibrotic agent allow to obtain a first score D named SD (before the administration) and a second score E named SE (after the administration).

Said SD and SE may be obtained through an algorithm equation, especially the function as defined above.

In a more particular embodiment, the method for assessing the efficacy of an anti-fibrotic agent comprises:
a) calculating SD obtained from the measures of levels of sVCAM and TSP-2 in a biological fluid sample isolated from a subject suffering from advanced liver fibrosis before an administration of an anti-fibrotic agent;
b) calculating SE obtained from the measures of levels of sVCAM and TSP-2 in a biological fluid sample isolated from the same subject after treatment with an antifibrotic compound;
c) comparing SE and SD;

If SE is higher than SD, then liver fibrosis worsens, the subject does not respond to the treatment with the anti-fibrotic agent or the treatment is not effective.

If SE is lower than SD, then liver fibrosis regresses and the subject responds to the treatment with the anti-fibrotic agent and the treatment is effective.

If SD is equal to SE, then liver fibrosis is stable.

The present invention also relates to a data-processing device comprising means for carrying one of the methods of the present invention as described above.

The present invention provides also a computer program product or a computer-readable storage medium comprising instructions which, when executed by a computer, cause the computer to carry out one of the methods of the present invention as described above.

The invention also relates to a kit for diagnosing advanced liver fibrosis or liver cirrhosis in a subject. Said kit comprises means for determining the levels of sVCAM and TSP-2. In a particular embodiment, said kit comprises specific antibodies, aptamers or peptides to measure sVCAM and TSP-2.

More particularly, said kit comprises an antibody, an aptamer or a peptide directed against sVCAM and an antibody, aptamer or peptide directed against TSP-2.

Antibodies directed against sVCAM may be any monoclonal, polyclonal and/or conjugated antibodies directed against sVCAM known in the art.

Antibodies directed against TSP-2 may be any monoclonal, polyclonal and/or conjugated antibodies directed against TSP-2 known in the art.

The kit of the invention may further comprise immunoassay standards and reagents. In a particular embodiment, the kit comprises at least one specific positive control for TSP-2 and/or at least one specific positive control for sVCAM.

Particularly, said positive control for TSP-2 comprises from 11 to 300 ng/ml of TSP-2 and said positive control for sVCAM comprises from 390 to 6190 ng/ml of sVCAM.

More particularly, the kit of the invention further comprises 3 positive controls for TSP-2 and 3 positive controls for sVCAM, wherein said positive controls for TSP-2 respectively comprise 30, 50 and 100 ng/ml of TSP-2; wherein said positive controls for sVCAM comprise 600, 800 and 1100 ng/ml of sVCAM.

The invention is further described with reference to the following, non-limiting, examples.

### EXAMPLES

### Example 1: Selection of Biological Markers

The selection of the biological markers was performed by logistic regression on a list of biological markers involved in the mechanism of action of liver fibrosis to select the most significant markers correlated with advanced liver fibrosis. 14 biological markers including soluble Vascular Cell Adhesion Molecule-1 (sVCAM), Thrombospondin 2 (TSP-2), miR34a, glycated haemoglobin (HbA1C), Age, platelets, aspartate aminotransferase (AST), alanine aminotransferase (ALT), tissue inhibitor of matrix metalloprotease-1 (TIMP1), N-terminal pro-peptide of collagen type III (P3nP) and Hyaluronic acid were included.

Individual markers were measured in serum of 1063 patients from phase 3 clinical trial RESOLVE-IT® (NCT) study. RESOLVE-IT® is a Multicenter, Randomized, Double-Blind, Placebo-Controlled Phase III Study (NCT02704403) to Evaluate the Efficacy and Safety of Elafibranor in Patients with Nonalcoholic Steatohepatitis (NASH) and fibrosis. Liver biopsy was performed to confirm the diagnosis and staging of liver fibrosis. Clinical data and blood samples were also collected for all patients. Liver fibrosis stage was provided by the analysis of the liver biopsy according to the Kleiner staging system. Liver fibrosis stages prevalence is reported in table 1.

TABLE 1
Liver fibrosis stages distribution in 1063 patients

| Liver Fibrosis Stage | 0 | 1 | 2 | 3 | 4 |
|---|---|---|---|---|---|
| Prevalence Fibrosis Stage (%) | 17.2 | 26.3 | 19.8 | 21.3 | 15.3 |

All markers were tested for their normality and those showing a non-gaussian curve were log transformed. From the 14 markers analyzed, 13 were log transformed and only TIMP-1 was used in a linear data format. After the Log 10 transformation of data (except for TIMP-1), a regression model was designed using advanced liver fibrosis and cirrhosis as the response variable using ToolPak analysis from Excel software. 8 biological markers were significantly correlated with advanced liver fibrosis and cirrhosis (p<0,01) and these markers were submitted to collinearity analysis. Hyaluronic acid and AST were excluded because of collinearity with TSP-2. A second regression model fitting was performed using liver fibrosis as the response variable and the 6 selected markers as the explanatory variables of advance fibrosis and cirrhosis. 3 markers that had a significant correlation with fibrosis status of our patients (p value<0.01) were selected (ALT was excluded because its p value was 0.153). A second correlation analysis was done to cluster covariates from the 2 markers selected (TSP-2 and sVCAM) and that were correlated with fibrosis status and demonstrated no collinearity between these circulating biomarkers by calculation of the variance inflation factor (VIF<0.2), a measure of the amount of multicollinearity in a set of multiple regression variables. This subset of 2 markers as variables was analyzed by logistic regression (using Excel Solver) to create algorithms from several combinations. These two markers, i.e. sVCAM and TSP-2, were identified as principal variables.

The same procedure on 1063 patients from phase 3 clinical trial RESOLVE-IT® (NCT) study was then assessed to develop a logistic regression that differentiates cirrhotic patients from the patients without Liver Cirrhosis. sVCAM and TSP-2 individual markers were also identified as principal variables for diagnosing liver cirrhosis.

Therefore, the combination of two circulating biomarkers, i.e. soluble Vascular Cell Adhesion Molecule-1 (sVCAM) and Thrombospondin 2 (TSP-2), provides an improved diagnosis of advanced liver fibrosis or liver cirrhosis compared to existing solutions and represents a valuable alternative to liver biopsy.

### Example 2: Diagnosis of Advanced Liver Fibrosis in a Human Subject with the Combination of sVCAM and TSP-2

The biomarkers were measured in patients' serum. TSP-2 was quantified by using Quantikine® ELISA Human Thrombospondin-2 Immunoassay (Catalog Number DTSP-20, BioTechne, France) according to the manufacturer's recommendations with positive controls set up to assess clinically relevant values for fibrosis. sVCAM dosage was performed by Quantikine® ELISA Human VCAM-1/CD106 Immunoassay. (Catalog Number DVC00. SVC00 and PDVC00 BioTechne), according to the manufacturer's recommendations with positive controls set up to assess clinically relevant values for fibrosis.

Three positive controls were developed to quantify TSP-2.

$$control 1 ( C 1 = 30 ng / mL TSP - 2 ) ,$$

$$control 2 ( C 2 = 50 ng / mL TSP - 2 )$$

$$control 3 ( C 3 = 100 ng / mL TSP - 2 ) .$$

C1 is made of serum of healthy volunteers' blood (Etablissement Français du Sang, France). C1 naturally contains TSP-2. C2 requires a dilution of C3 to obtain a 50 ng/ml TSP-2 diluted in serum of healthy volunteers. C3 is prepared from lyophilized recombinant human TSP-2 diluted in serum of healthy volunteers.

Three positive controls were also developed to quantify sVCAM with control 1 (C′1=600 ng/ml sVCAM), control 2 (C′2=800 ng/mL sVCAM) and control 3 (C′3=1 100 ng/ml sVCAM).

C′1 is made of serum of healthy volunteers (Etablissement Français du Sang, France). C′1 naturally contains sVCAM. C′2 requires a dilution of C′3 to obtain an 800 ng/ml sVCAM in serum of healthy volunteers. C′3 is prepared from lyophilized recombinant human sVCAM diluted in serum of healthy volunteers.

All the controls are aliquoted and stored at −80° C. until use. Positive controls are processed similarly to sample specimen and assayed in duplicate.

A first score SA score is defined with k=−30.00, a=7.250 and b=6.921.

To differentiate patients with advanced liver fibrosis from patients without advanced liver fibrosis, the cut-off co1 for the diagnostic of advanced liver fibrosis is set at 0.3471.

ELF and FIB-4 were calculated according to the literature. SA score was then compared to ELF and FIB4 tests and as well as with individual biomarkers by the mean of their Area Under the Receiver Operating characteristic Curve (AUROC) for the diagnostic of advanced liver fibrosis (Table 2).

TABLE 2
Diagnostic metrics of SA score (sVCAM and TSP-2) with individual biomarkers and
scores like FIB4 and ELF test on Advanced Liver Fibrosis diagnosis. AUROC: Area
Under the Receiver Operating characteristic Curve, SE: standard Error, CI: Confidence
Interval, Sens. (sensitivity), Spec. (specificity), PLR (Positive Likelihood
Ratio), NLR (Negative Likelihood Ratio), AF (Advanced liver fibrosis), Acc. (Accuracy),
PPV (Positive Predictive Value), NPV (Negative Predictive Value). 674 patients
have a fibrosis <3 and 389 patients have a fibrosis ≥3.

| | Diagnostic metrics Advanced Liver Fibrosis | | | | | |
|---|---|---|---|---|---|---|
| | SA: [sVCAM and TSP-2] | | ELF | | FIB4 | |
| | Cut-off (co1) | | | | | |
| | 0.3383 | | 9.574 | | 1.312 | |
| | Data | | | | | |
| | Value | 95% CI | Value | 95% CI | Value | 95% CI |
| AUROC | 0.897 | 0.8776-0.9163 | 0.8333 | 0.8079-0.8588 | 0.7911 | 0.7626-0.8196 |
| Sens. (%) | 81.75 | 77.54-85.46 | 75.84 | 71.27-80.01 | 72.24 | 67.50-76.63 |
| Spec. (%) | 81.75 | 78.62-84.60 | 75.82 | 72.40-79.00 | 72.43 | 68.88-75.78 |
| PLR | 4.48 | 3.79-5.29 | 3.14 | 2.71-3.62 | 2.62 | 2.28-3.01 |
| NLR | 0.22 | 0.18-0.28 | 0.32 | 0.27-0.38 | 0.38 | 0.32-0.45 |
| AF Prevalence (%) | 36.59 | 33.69-39.57 | 36.59 | 33.69-39.57 | 36.7 | 33.79-39.68 |
| PPV (%) | 72.11 | 68.64-75.33 | 64.41 | 61.02-67.66 | 60.3 | 56.97-63.54 |
| NPV (%) | 88.59 | 86.24-90.57 | 84.46 | 81.93-86.69 | 81.82 | 79.20-84.17 |
| Acc. (%) | 81.75 | 79.29-84.03 | 75.82 | 73.13-78.37 | 72.36 | 69.56-75.03 |

The diagnosis with SA score and co1 gives rise—the highest performances as shown by the AUC relative—individuals biomarkers and scores like ELF test and FIB4-diagnose advanced liver fibrosis. The Area Under the Receiver Operating characteristic Curve (AUROC) reaches 0.897 with SA score (sVCAM and TSP-2) whereas the AUC of the ELF test is equal to 0.8333, and the AUC of FIB4 is equal to 0.7911.

Diagnostic metrics (total accuracy, sensitivity, specificity, positive predictive value/PPV, negative predictive value/NPV, positive likelihood ratio/LR+ and negative likelihood ratio/LR−) are also provided in Table 2, with 95% CI calculated with the asymptotic formula based on the normal approximation—the binomial distribution. The diagnostic metrics allows the comparison of the three assays: the method of the invention with S score (sVCAM and TSP-2), with ELF test and FIB4.

The detailed comparison highlights that SA score is a more sensitive diagnostic assay for advanced liver fibrosis compared-ELF test and FIB4 (Sensitivity=81.75% for the method of the invention versus 75.84% with ELF and 72.24% with FIB4). The superiority of SA score (sVCAM and TSP-2) was also observed on specificity (81.75% for the method of the invention versus 75.82% with ELF test and 72.43% with FIB4), PLR was also greater with SA score than ELF test and FIB4 (4.48 vs 3.14 with ELF test and 2.62 with FIB4) and NLR was lesser with SA score than ELF test and FIB4 (0.22 vs 0.32 with ELF and 0.38 with FIB4), PPV was higher with SA than ELF test and FIB4 (72.11% versus 64.41% with ELF and 60.3 with FIB4), and NPV was higher with SA than ELF test and FIB4 (88.59% versus 84.46% with ELF and 81.82% with FIB4), and therefore the global performance of the method of the invention is the greatest in term of Accuracy for SA score (81.75% versus 75.82% with ELF and 72.36% with FIB4).

TABLE 3
Statistical difference between AUROC fibrosis tests to
diagnose Advanced Liver Fibrosis. Differences in AUROC
were assessed using the DeLong test (Ref: DeLong, E.
R., et al. Comparing the Areas under Two or More Correlated
Receiver Operating Characteristic Curves: A Nonparametric
Approach. Biometrics 1988, 44: 837-845).

| | | | p value vs. |
|---|---|---|---|
| AUROC comparison, | | p = 2*(1 − | SA (sVCAM |
| Advanced Liver Fibrosis | AUROC | Normdist(z)) | and TSP-2) |
| SA score (sVCAM and | 0.897 | — | — |
| TSP-2) | | | |
| ELF | 0.8333 | 9.48651E−05 | **** |
| FIB4 | 0.7911 | 1.66953E−09 | **** |

The statistical difference of the diagnostic performance of SA score was then assessed relatively to the two other scores: ELF test and FIB4. As shown in table 3, the AUROC of the method of the invention is significantly different from ELF (p<0.0001) and FIB4 (p<0.0001). The SA score presents the highest AUROC to diagnose Advanced Liver Fibrosis compared to ELF test and FIB4.

To differentiate patients with advanced liver fibrosis from patients without advanced liver fibrosis, the cut-off for the diagnostic of advanced liver fibrosis is set at 0.204 (co1-1) at 90% of sensitivity (Data metrics Table 4). Alternatively, the cut-off is set at 0.4852 (co1-2) at 90% specificity (Data metrics Table 5).

TABLE 4
Diagnostic metrics of SA score (sVCAM and TSP-2) with individual biomarkers and scores like FIB4
and ELF test on Advanced Liver Fibrosis diagnosis at 90% of sensitivity. AUROC: Area Under the
Receiver Operating characteristic Curve, SE: standard Error, CI: Confidence Interval, Sens. (sensitivity),
Spec. (specificity), PLR (Positive Likelihood Ratio), NLR (Negative Likelihood Ratio), AF (Advanced
liver fibrosis), Acc. (Accuracy), PPV (Positive Predictive Value), NPV (Negative Predictive Value).
674 patients have a fibrosis <3 and 389 patients have a fibrosis ≥3.

| | Diagnostic metrics Advanced Liver Fibrosis | | | | | |
|---|---|---|---|---|---|---|
| | SA: [sVCAM, TSP-2] | | ELF | | FIB4 | |
| | Cut-off (co1-1) | | | | | |
| | 0.204 | | 9.022 | | 0.8279 | |
| | Data | | | | | |
| | Value | 95% CI | Value | 95% CI | Value | 95% CI |
| AUROC | 0.897 | 0.8776-0.9163 | 0.8333 | 0.8079-0.8588 | 0.7911 | 0.7626-0.8196 |
| Sens. (%) | 90.00 | 86.55-92.77 | 90.00 | 86.55-92.77 | 90.00 | 86.55-92.77 |
| Spec. (%) | 69.14 | 65.50-72.61 | 50.15 | 46.31-53.99 | 37.41 | 33.73-41.19 |
| PLR | 2.92 | 2.59-3.28 | 1.8 | 1.66-1.96 | 1.44 | 1.34-1.54 |
| NLR | 0.15 | 0.11-0.20 | 0.2 | 0.15-0.27 | 0.27 | 0.20-0.37 |
| AF Prevalence (%) | 36.59 | 33.69-39.57 | 36.59 | 33.69-39.57 | 36.7 | 33.79-39.68 |
| PPV (%) | 62.72 | 59.93-65.43 | 51.02 | 48.95-53.08 | 45.45 | 43.79-47.13 |
| NPV (%) | 92.28 | 89.83-94.17 | 89.66 | 86.44-92.18 | 86.55 | 82.47-89.80 |
| Acc. (%) | 76.76 | 74.11-79.27 | 64.72 | 61.77-67.60 | 56.7 | 53.65-59.71 |

TABLE 5
Diagnostic metrics of S score (sVCAM and TSP-2) with individual biomarkers and scores like
FIB4 and ELF test on Advanced Liver Fibrosis diagnosis at 90% of specificity. AUROC: Area
Under the Receiver Operating characteristic Curve. SE: standard Error. CI: Confidence
Interval. Sens. (sensitivity). Spec. (specificity). PLR (Positive Likelihood Ratio). NLR
(Negative Likelihood Ratio). AF (Advanced liver fibrosis). Acc. (Accuracy). PPV (Positive
Predictive Value). NPV (Negative Predictive Value). 674 patients have a fibrosis <3
and 389 patients have a fibrosis ≥3. Data metrics with SA score with co1, co1-1 and
co1-2 are better than ELF test and FIB4 on the diagnosis of advanced liver fibrosis.

| | Diagnostic metrics Advanced Liver Fibrosis | | | | | |
|---|---|---|---|---|---|---|
| | SA: [sVCAM, TSP-2] | | ELF | | FIB4 | |
| | Cut-off (co1-2) | | | | | |
| | 0.4852 | | 10.11 | | 1.869 | |
| | Data | | | | | |
| | Value | 95% CI | Value | 95% CI | Value | 95% CI |
| AUROC | 0.897 | 0.8776-0.9163 | 0.8333 | 0.8079-0.8588 | 0.7911 | 0.7626-0.8196 |
| Sens. (%) | 70.95 | 66.16-75.42 | 55.27 | 50.18-60.28 | 47.81 | 42.76-52.91 |
| Spec. (%) | 90 | 87.55-92.21 | 90 | 87.55-92.21 | 90 | 87.55-92.21 |
| PLR | 7.14 | 5.64-9.04 | 5.56 | 4.36-7.10 | 4.81 | 3.75-6.18 |
| NLR | 0.32 | 0.28-0.38 | 0.5 | 0.44-0.56 | 0.58 | 0.53-0.64 |
| AF Prevalence (%) | 36.59 | 33.69-39.57 | 36.59 | 33.69-39.57 | 36.7 | 33.79-39.68 |
| PPV (%) | 80.47 | 76.49-83.91 | 76.24 | 71.54-80.38 | 73.52 | 68.38-78.09 |
| NPV (%) | 84.31 | 82.11-86.28 | 77.72 | 75.70-79.62 | 74.94 | 73.05-76.74 |
| Acc. (%) | 83.07 | 80.67-85.27 | 77.33 | 74.69-79.81 | 74.6 | 71.87-77.19 |

A second SA score is defined with k=−34, a=8.6 and b=6.7

A third SA score is defined with k=−32.227, a=8.640 and b=5.750

A fourth SA score is defined with k=−27.200, a=5.900 and b=7.700.

These models give rise to an AUROC not significantly different from the first SA score to diagnose advanced liver fibrosis (Table 6).

TABLE 6
AUROC values according to the different algorithms and variation of
coefficient in a selected range to diagnose advanced liver fibrosis.

| SA model | Model 1 | Model 2 | Model 3 | Model 4 |
|---|---|---|---|---|
| Constant Coef. (k) | −30.00 | −34.000 | −32.227 | −27.200 |
| TSP-2 Coef. (b) | 6.921 | 6.700 | 5.750 | 7.700 |
| sVCAM-1 Coef. (a) | 7.250 | 8.600 | 8.640 | 5.900 |
| AUROC | 0.8970 | 0.8964 | 0.8951 | 0.8959 |
| 95% confidence | 0.8857-0.9225 | 0.8821-0.9197 | 0.8849-0.9219 | 0.8856-0.9224 |
| interval | | | | |
| Std. Error | 0.009388 | 0.009586 | 0.009424 | 0.009391 |
| p = 2*(1 − | — | 0.81149585 | 0.95803213 | 0.99399133 |
| Normdist(z)) | | | | |
| Interpretation | — | NS | NS | NS |

### Example 3: Diagnosis of Liver Cirrhosis in a Human Subject with the Combination of sVCAM and TSP-2

To differentiate patients with Liver Cirrhosis from patients without Liver Cirrhosis (F<4). the cut-off co 2 for the diagnostic of Liver Cirrhosis was set at 0.6073.

ELF and FIB-4 were calculated according to the literature. The SA score was then compared to ELF and FIB4 tests and as well as with individual biomarkers by the mean of their Area Under the Receiver Operating characteristic Curve (AUROC) for the diagnostic of Liver Cirrhosis (Table 6).

TABLE 7
Diagnostic metrics of SA score (sVCAM and TSP-2) with individual biomarkers and
scores like FIB4 and ELF test on Liver Cirrhosis diagnosis. AUROC: Area Under
the Receiver Operating characteristic Curve. SE: standard Error. CI: Confidence
Interval. Sens. (sensitivity). Spec. (specificity). PLR (Positive Likelihood Ratio).
NLR (Negative Likelihood Ratio). C Prevalence (Liver Cirrhosis Prevalence). Acc.
(Accuracy). PPV (Positive Predictive Value). NPV (Negative Predictive Value).
900 patients have a fibrosis <4 and 163 patients have a fibrosis ≥4.

| | Diagnostic metrics Liver Cirrhosis | | | | | |
|---|---|---|---|---|---|---|
| | SA: [sVCAM. TSP-2] | | ELF | | FIB4 | |
| | Cut-off (co2) | | | | | |
| | 0.6073 | | 9.966 | | 1.557 | |
| | Data | | | | | |
| | Value | 95% CI | Value | 95% CI | Value | 95% CI |
| AUROC | 0.9111 | 0.8911-0.9311 | 0.8672 | 0.84-0.8944 | 0.8103 | 0.7759-0.8447 |
| Sens. (%) | 82.82 | 76.14-88.27 | 77.91 | 70.76-84.03 | 73.62 | 66.16-80.20 |
| Spec. (%) | 82.89 | 80.27-85.29 | 77.89 | 75.03-80.56 | 73.69 | 70.68-76.55 |
| PLR | 4.84 | 4.13-5.68 | 3.52 | 3.04-4.08 | 2.8 | 2.43-3.23 |
| NLR | 0.21 | 0.15-0.29 | 0.28 | 0.21-0.38 | 0.36 | 0.28-0.46 |
| C Prevalence (%) | 15.33 | 13.22-17.64 | 15.33 | 13.22-17.64 | 15.38 | 13.26-17.69 |
| PPV (%) | 46.71 | 42.76-50.71 | 38.96 | 35.51-42.51 | 33.71 | 30.59-36.97 |
| NPV (%) | 96.38 | 95.00 to 97.39 | 95,12 | 93.57-96.30 | 93.89 | 92.22-95.22 |
| Acc. (%) | 82.88 | 80.48 to 85.10 | 77.89 | 75.28-80.35 | 73.68 | 70.92-73.31 |

The diagnosis with SA score and co2 and gives rise to the highest performances as shown by the AUROC relative to individuals biomarkers and scores like ELF test and FIB4 to diagnose Liver Cirrhosis. The Area Under the Receiver Operating characteristic Curve (AUROC) reaches 0.9111 with the SA score (sVCAM and TSP-2) whereas the AUROC of the ELF test is equal to 0.8672, and the AUROC of FIB4 is equal to 0.8103 (Table 7).

Diagnostic metrics (total accuracy. sensitivity. specificity. positive predictive value/PPV. negative predictive value/NPV. positive likelihood ratio/LR+ and negative likelihood ratio/LR−) are also provided in table 7 with 95% CI calculated with the asymptotic formula based on the normal approximation to the binomial distribution. The diagnostic metrics allow the comparison of the three assays: the method of the invention with SA score (sVCAM and TSP-2) with ELF test and FIB4.

The detailed comparison highlights that SA score is a more sensitive diagnostic assay for Liver Cirrhosis compared to ELF test and FIB4 (Sensitivity=82.82% for the method of the invention versus 77.91% with ELF and 73.62% with FIB4). The superiority of SA score (sVCAM and TSP-2) was also observed on specificity (82.89% for the method of the invention versus 77.89% with ELF test and 73.69% with FIB4). PLR was also greater with SA score than ELF test and FIB4 (4.84 vs 3.92 with ELF test and 2.8 with FIB4) and NLR was lesser with SA score than ELF test and FIB4 (0.21 vs 0.28 with ELF and 0.36 with FIB4). PPV was higher with SA than ELF test and FIB4 (46.71% versus 38.96% with ELF and 33.71% with FIB4), and NPV was higher with SA than ELF test and FIB4 (96.38% versus 95.12% with ELF and 93.89% with FIB4). Therefore, the global performance of the method of the invention is greater in term of Accuracy for SA score (82.88% versus 77.89% with ELF and 73.68% with FIB4).

TABLE 8
Statistical difference between AUROC fibrosis
tests to diagnose Liver Cirrhosis. Differences
in AUROC were assessed using the DeLong test.

| | | | p value |
|---|---|---|---|
| AUROC comparison. | | p = 2*(1 − | vs. SA (sVCAM |
| Liver Cirrhosis | AUROC | Normdist(z)) | and TSP-2) |
| SA score | 0.9111 | — | — |
| ELF | 0.8672 | 0.002997075 | ** |
| FIB4 | 0.8103 | 6.91666E−07 | **** |

The statistical difference of the diagnostic performance of SA score was then assessed relatively to the two other scores: ELF test and FIB4. As shown in Table 8, the AUROC of the method of the invention is significantly different from ELF (p<0.01) and FIB4 (p<0.0001). The SA score presents the highest AUROC to diagnose Liver Cirrhosis compared to ELF test and FIB4.

To differentiate patients with Liver Cirrhosis from patients without Liver Cirrhosis, the cut-off for the diagnostic of Liver Cirrhosis is set at 0.5110 (co2-1) at 90% of sensitivity (Data metrics Table 9). Alternatively, the cut-off is set at 0.7791 (co2-2) at 90% specificity (Data metrics Table 10). In both cases data metrics are better with SA score than ELF test and FIB4.

TABLE 9
Diagnostic metrics of the combination of sVCAM and TSP-2 with individual biomarkers
and scores like FIB4 and ELF test on Cirrhosis diagnosis at 90% of sensitivity.
AUROC: Area Under the Receiver Operating characteristic Curve. SE: standard Error.
CI: Confidence Interval. Sens. (sensitivity). Spec. (specificity). PLR (Positive
Likelihood Ratio). NLR (Negative Likelihood Ratio). C Prevalence (Cirrhosis prevalence).
Acc. (Accuracy). PPV (Positive Predictive Value). NPV (Negative Predictive Value).
900 patients have a fibrosis <4 and 163 patients have a fibrosis = 4.

| | Diagnostic metrics Liver Cirrhosis | | | | | |
|---|---|---|---|---|---|---|
| | SA: [sVCAM. TSP-2] | | ELF | | FIB4 | |
| | Cut-off (co2-1) | | | | | |
| | 0.5110 | | 9.56 | | 1.1 | |
| | Data | | | | | |
| | Value | 95% CI | Value | 95% CI | Value | 95% CI |
| AUROC | 0.9111 | 0.8911-0.9311 | 0.8672 | 0.84-0.8944 | 0.8103 | 0.7759-0.8447 |
| Sens. (%) | 90.00 | 84.55-94.28 | 90.00 | 84.55-94.28 | 90.00 | 84.55-94.28 |
| Spec. (%) | 76.56 | 73.65-79.29 | 64.78 | 61.56-67.90 | 50.84 | 47.51-54.16 |
| PLR | 3.85 | 3.38-4.37 | 2.56 | 2.31-2.84 | 1.83 | 1.69-1.99 |
| NLR | 0.13 | 0.08-0.20 | 0.15 | 0.09-0.24 | 0.19 | 0.13-0.31 |
| C Prevalence (%) | 15.33 | 13.22-17.64 | 15.33 | 13.22-17.64 | 15.38 | 13.26-17.69 |
| PPV (%) | 41.06 | 37.99-44.20 | 31.68 | 29.51-33.93 | 25 | 23.46-26.60 |
| NPV (%) | 97.73 | 96.43-98.56 | 97.33 | 95.80-98.31 | 96.61 | 94.69-97.85 |
| Acc. (%) | 78.65 | 76.06-81.07 | 68.67 | 65.79-71.45 | 56.89 | 53.84-59.89 |

TABLE 10
Diagnostic metrics of the combination of sVCAM and TSP-2 with individual biomarkers
and scores like FIB4 and ELF test on Liver Cirrhosis diagnosis at 90% of specificity.
AUROC: Area Under the Receiver Operating characteristic Curve. SE: standard Error.
CI: Confidence Interval. Sens. (sensitivity). Spec. (specificity). PLR (Positive Likelihood
Ratio). NLR (Negative Likelihood Ratio). C Prevalence (Liver Cirrhosis prevalence).
Acc. (Accuracy). PPV (Positive Predictive Value). NPV (Negative Predictive Value).
900 patients have a fibrosis <4 and 163 patients have a fibrosis = 4.

| | Diagnostic metrics Liver Cirrhosis | | | | | |
|---|---|---|---|---|---|---|
| | SA: [sVCAM and TSP-2.] | | ELF | | FIB4 | |
| | Cut-off (co2-2) | | | | | |
| | 0.7791 | | 10.48 | | 2.295 | |
| | Data | | | | | |
| | Value | 95% CI | Value | 95% CI | Value | 95% CI |
| AUROC | 0.9111 | 0.8911-0.9311 | 0.8672 | 0.84-0.8944 | 0.8103 | 0.7759-0.8447 |
| Sens. (%) | 69.33 | 61.63-76.30 | 59.51 | 51.55-67.12 | 44.79 | 37.00-52.76 |
| Spec. (%) | 90 | 87.85-91.88 | 90 | 87.85-91.88 | 90 | 87.85-91.88 |
| PLR | 6.93 | 5.56-8.65 | 5.95 | 4.71-7.51 | 4.51 | 3.48-5.86 |
| NLR | 0.34 | 0.27-0.43 | 0.45 | 0.37-0.54 | 0.61 | 0.53-0.71 |
| C Prevalence (%) | 15.33 | 13.22-17.64 | 15.33 | 13.22-17.64 | 15.38 | 13.26-17.69 |
| PPV (%) | 55.67 | 50.16-61.03 | 51.87 | 46.05-57.65 | 45.06 | 38.73-51.66 |
| NPV (%) | 94.19 | 92.78-95.33 | 92.47 | 91.05-93.67 | 89.98 | 88.64-91.17 |
| Acc. (%) | 86.83 | 84.65-88.81 | 85.32 | 83.05-87.40 | 83.11 | 80.72-85.32 |

In addition, data metrics with SA score with co2-1 (Table 9) and co2-2 (Table 10) are both better than ELF test and FIB4 on the diagnosis of Liver Cirrhosis.

The other models give rise to an AUROC not significantly different from the first score to diagnose liver cirrhosis (Table 11).

TABLE 11
AUROC values according to the different algorithms and variation
of coefficient in a selected range to diagnose liver cirrhosis.

| | Model 1 | Model 2 | Model 5 | Model 4 |
|---|---|---|---|---|
| Constant Coef. (k) | −30.00 | −34.000 | −32.227 | −27.200 |
| TSP-2 Coef. (b) | 7.250 | 6.700 | 5.750 | 7.700 |
| sVCAM-1 Coef. (a) | 6.921 | 8.600 | 8.640 | 5.900 |
| AUROC | 0.9111 | 0.911 | 0.9096 | 0.9088 |
| 95% confidence interval | 0.8911-0.9311 | 0.8922-0.9319 | 0.8894-0.9298 | 0.8884-0.9291 |
| Std. Error | 0.0102 | 0.01012 | 0.0103 | 0.01038 |
| p = 2*(1 − Normdist(z)) | | 0.9445147553 | 0.9175837599 | 0.8744213131 |
| Interpretation | — | NS | NS | NS |

1-20. (canceled)

21. An in vitro method for diagnosing or prognosing advanced liver fibrosis or liver cirrhosis in a subject, comprising
(a) measuring the circulating levels of Soluble Vascular Cell Adhesion Molecule-1 (sVCAM) and Thrombospondin 2 (TSP-2) in a biological fluid sample isolated from said subject;
(b) comparing the levels of sVCAM and TSP-2 with reference levels of sVCAM and TSP-2, wherein the comparison between measured levels and reference levels is indicative of the presence or absence of advanced liver fibrosis or liver cirrhosis.

22. The method according to claim 21, wherein the biological fluid sample is a saliva sample, an interstitial liquid sample, an urine sample, a blood sample, a plasma sample or a serum sample.

23. The method according to claim 22, wherein in step (b) a score A (SA) is compared to a cut-off value, said SA being obtained from the levels of sVCAM and TSP-2 measured in step (a), said cut-off value being obtained from reference levels of sVCAM and TSP-2, said SA and cut-off value being calculated using an algorithm equation.

24. The method according to claim 23, wherein the SA is calculated through the following algorithm equation:
$$SA = {e}^{y} / ( 1 + {e}^{y} ) with$$
$$y = k + a \times A + b \times B$$
A is the level of sVCAM in log 10 ng/ml;
B is the level of TSP-2 in log 10 ng/ml;
k is the constant of the algorithm equation;
a is a coefficient associated to the level of sVCAM;
b is a coefficient associated to the level of TSP-2;
and wherein:
k is a number between −37.23 and −27.54 or is-30.000;
a is a number between 5.432 and 8.646 or is 7.250;
b is a number between 5.746 and 7.727 or is 6.921.

25. The method according to claim 23, wherein SA higher than a cut-off value co1 is indicative of advanced liver fibrosis and co1 is between 0.200 and 0.500 or co1 is equal to 0.3383.

26. The method according to claim 23, wherein SA higher than a cut-off value co2 is indicative of a liver cirrhosis and co2 is between 0.510 and 0.790 or is equal to 0.6073.

27. An in vitro method for monitoring the progression of liver fibrosis in a subject, comprising the steps of:
(a) measuring the circulating levels sVCAM and TSP-2 in a biological fluid sample isolated from said subject, and
(b) comparing said levels with levels of sVCAM and TSP-2 previously measured in the same subject.

28. The method according to claim 27, wherein in step (b) a score C (SC) is compared to a score B (SB), SC being a score obtained from the measured levels of sVCAM and TSP-2 of step (a) and SB being a score obtained from levels of sVCAM and TSP-2 previously measured in the same subject, SB and SC being calculated using an algorithm equation; and wherein
an increase of SC compared to SB indicates the progression of liver fibrosis;
a decrease of SC compared to SB indicates the regression of liver fibrosis;
no difference between SC and SB indicates a stable liver fibrosis.

29. The method according to claim 28, wherein SC is measured at least 3 months after the measurement of SB, between 3 months and 10 years after measurement of SB, or between 3 months and 2 years after measurement of SB.

30. The method according to claim 28, wherein SC or SB are calculated through the following algorithm equation:
$$SC or SB = {e}^{y} / ( 1 + {e}^{y} ) with$$
$$y = k + a \times A + b \times B$$
A is the level of sVCAM in log 10 ng/ml;
B is the level of TSP-2 in log 10 ng/ml;
k is the constant of the algorithm equation;
a is a coefficient associated to the level of sVCAM;
b is a coefficient associated to the level of TSP-2;
and wherein:
k is a number between −37.23 and −27.54 or is-30.000;
a is a number between 5.432 and 8.646 or is 7.250;
b is a number between 5.746 and 7.727 or is 6.921.

31. A method of treating advanced liver fibrosis or liver cirrhosis in a subject comprising diagnosing a subject as suffering from advanced liver fibrosis or liver cirrhosis according to the method of claim 21 and administering an agent selected from the group consisting of pegbelfermin, Cenicriviroc, Dapagliflozin, Dulaglutide, Empagliflozin, Fenofibrate, Lanifibranor, Liraglutide, obeticholic acid, Pioglitazone, Resmetirom, saroglitazar magnesium, Seladelpar, Semaglutide, Sitagliptin, TERN-101, TERN-201, Tropifexor, Ambrisentan, BMS-963272, BMS-986251, BMS-986263, HepaStem, LYS006, MET409, MET642 and orlistat to a subject having advanced liver fibrosis or liver cirrhosis.

32. A method for assessing the efficacy of an anti-fibrotic agent in treating advanced liver fibrosis or liver cirrhosis, comprising
(a) measuring the circulating levels of sVCAM and TSP-2 in a biological fluid sample isolated from a subject suffering from advanced liver fibrosis, wherein said subject has been administered an anti-fibrotic agent before said measure, and
(b) comparing said levels of sVCAM and TSP-2 with the levels of sVCAM and TSP-2, previously measured before administration of the anti-fibrotic agent to the same subject to assess the efficacy of said anti-fibrotic agent.

33. The method according to claim 32, wherein in step (b) a score E (SE) is compared to a score D (SD),
SD being a score obtained from the levels of sVCAM and TSP-2 measured before administration of an anti-fibrotic agent to the subject,
SE being a score obtained from the levels of sVCAM and TSP-2 measured after administration of an anti-fibrotic agent to the subject, and
SD and SE being calculated through an algorithm equation; and
wherein a decrease of SE compared to SD indicates the efficacy of the anti-fibrotic agent.

34. The method according to claim 33, wherein SE and SD are calculated through the following algorithm equation:
$$SE or SD = {e}^{y} / ( 1 + {e}^{y} ) with$$
$$y = k + a \times A + b \times B$$
A is the level of sVCAM in log 10 ng/ml;
B is the level of TSP-2 in log 10 ng/mL;
k is the constant of the algorithm equation;
a is a coefficient associated to the level of sVCAM;
b is a coefficient associated to the level of TSP-2;
and wherein:
k is a number between −37.23 and −27.54 or is −30.000;
a is a number between 5.432 and 8.646 or is 7.250;
b is a number between 5.746 and 7.727 or is 6.921.

35. A kit for diagnosing advanced liver fibrosis or liver cirrhosis in a subject, said kit comprising means for determining the levels of sVCAM and TSP-2, wherein the kit comprises at least one specific positive control for TSP-2 and/or at least one specific positive control for sVCAM.

36. The kit according to claim 35, comprising an antibody or an aptamer or a peptide directed against sVCAM and an antibody or an aptamer or a peptide directed against TSP-2.

37. A computer assisted program comprising instructions that, when executed by a processor/processing means, cause the processor/processing means to:
receive measured levels of sVCAM and TSP-2;
calculate a SA score from these measured levels, from the mathematical function
$$SA = {e}^{y} / ( 1 + {e}^{y} ) with$$
$$y = k + a \times A + b \times B$$
A is the level of sVCAM in log 10 ng/ml;
B is the level of TSP-2 in log 10 ng/ml;
k is the constant of the algorithm equation;
a is a coefficient associated to the level of sVCAM;
b is a coefficient associated to the level of TSP-2;
and wherein:
k is a number between −37.23 and −27.54 or is −30.000;
a is a number between 5.432 and 8.646 or is 7.250;
b is a number between 5.746 and 7.727 or is 6.921; and
assign the subject into the group of subjects having advanced liver fibrosis or liver cirrhosis upon the calculated score compared to predetermined cutoff values.

38. A data-processing device comprising means for carrying the method of claim 21.

39. A computer program product comprising instructions which, when the program is executed by a computer, cause the computer to carry out the method of claim 21.

40. A computer-readable storage medium comprising instructions which, when executed by a computer, cause the computer to carry out the method of claim 21.
